(19) United States
(12) Patent Application Publication
(10) Pub. No.: US 2022/0022968 A1
(43) Pub. Date: Jan. 27, 2022

(54) COMPUTER INPUT METHOD USING A DIGITIZER AS AN INPUT DEVICE

(71) Applicant: Think Surgical, Inc., Fremont, CA (US)

(72) Inventor: Pierre Brisson, Fremont, CA (US)

(73) Assignee: Think Surgical, Inc., Fremont, CA (US)

(21) Appl. No.: 17/311,444

(22) PCT Filed: Nov. 27, 2019
(86) PCT No.: PCT/US2019/063642

§ 371 Date: Jun. 7, 2021

Related U.S. Application Data
(60) Provisional application No. 62/773,738, filed on Nov. 30, 2018.

(51) Int. Cl. A61B 34/20; A61B 34/30; A61B 34/10; G06F 3/01

(52) U.S. Cl. CPC A61B 34/20; A61B 34/30; A61B 34/10; G06F 3/017; A61B 2090/3945; A61B 2034/2057; A61B 2034/105; A61B 2034/102; A61B 2034/2068

(57) ABSTRACT

A system and method is provided that supplies computer inputs using a tracked pointer as an input device during computer-assisted surgery. The system and method provides input or feedback to a computer-assisted surgical device. The system and method utilizes a tracking system for tracking a set of devices in an operating room. The set of devices includes a tracked surgical device that performs one or more tasks on a patient, a tracked display device that displays data related to the operation of the surgical device, and a tracked pointer to interact with the tracked display device and input data into a computing system associated with the tracking system and the tracked display device. The tracked pointer inputs data into the computing system based on the relative position and orientation (POSE) of the tracked pointer relative to the POSE of the tracked display device.

### RELATED APPLICATIONS

This application claims priority benefit of U.S. Provisional Application Ser. No. 62/773,738 filed 30 Nov. 2019, the contents of which are hereby incorporated by reference.

### TECHNICAL FIELD

The present invention generally relates to computer-assisted surgery, and more particularly to a system and method to provide computer inputs using a tracked pointer as an input device during computer-assisted surgery.

### BACKGROUND

Computer-assisted surgery is becoming more commonplace in the operating room (OR) because the clinical outcomes associated therewith are substantially better than manual or conventional techniques. Examples of computer-assisted surgical systems include the ROSA® Surgical System (Zimmer Biomet, Warsaw, Ind.) to aid with brain surgery, the da Vinci® Surgical System (Intuitive Surgical, Inc. Sunnyvale, Calif.) to aid with soft-tissue procedures, and the TSOLUTION ONE® Surgical System (THINK Surgical, Fremont, Calif.) to aid with orthopedic surgery.

Most computer-assisted surgical systems generally include a computer, a surgical device, and a display device. The display device may display workflow instructions to the user to guide the user through the surgical procedure. The workflow instructions may require input or feedback from the user during different stages of the procedure. For instance, the workflow may require the user to acknowledge the completion of a particular task (e.g., registration, calibration) before permitting the surgical system to proceed to a subsequent task. The means for inputting or providing the feedback to the system has relied on hand-held controllers or touch-screen monitors. However, there are several drawbacks to these devices. With the touch-screen monitor, a member of the surgical team is bound to the location of the monitor or has to continually move to the monitor to touch the monitor and provide the input. The sterility of the monitor is also important and often involves the use of a sterile drape covering the monitor. In some instances, the drape is soiled and may be difficult to see through. With the hand-held controller, the user loses the use of a hand and has to continually put the controller down to wield other surgical instruments. The hand-held controllers are also physically wired to the surgical system for safety, which might limit the mobility of the user to the length of the wires.

In light of the foregoing, there exists a need for a system and method to provide input or feedback to a computer-assisted surgical device in a more efficient and effective manner.

### SUMMARY

A surgical system is provided that includes a tracking system for tracking a set of devices in an operating room. The set of devices includes a tracked surgical device that performs one or more tasks on a patient, a tracked display device that displays data related to the operation of the surgical device, and a tracked pointer to interact with the tracked display device and input data into a computing system associated with the tracking system and the tracked display device. The tracked pointer inputs data into the computing system based on the relative position and orientation (POSE) of the tracked pointer relative to the POSE of the tracked display device. The inputted data affects at least one of: the displayed data related to the operation of the surgical device, or the operation of the surgical device itself.

A method is provided for inputting data into a surgical system by pointing a tracked pointer towards a tracked display device to activate an input data mode in the computing system in order to adjust at least one of a position or orientation of the tracked pointer to adjust a position of a cursor displayed on the display device, or for selecting one or more selections on the display device by way of: a gesture performed by wielding the tracked pointer, or by activating a selection function on the tracked pointer.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 depicts a robotic surgical system having a tracked pointer, tracked display device, and tracking system to permit a user to provide input data to the surgical system in accordance with embodiments of the invention;

FIG. 2 depicts a surgical system having a tracked hand-held surgical device, a tracked pointer, tracked display device, and tracking system to permit a user to provide input data to the surgical system in accordance with embodiments of the invention;

FIG. 3 depicts a robotic surgical system having a tracked device with an attached tracking array to permit a tracking system to track the display device in accordance with embodiments of the invention; and

FIG. 4 depicts a robotic surgical system having a tracked pointer and a mechanically tracked display device to permit a user to provide input data to the surgical system in accordance with embodiments of the invention.

### DETAILED DESCRIPTION

The present invention has utility as a system and method to provide input or feedback to a computer-assisted surgical device in an efficient and effective manner.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from that embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “tracked pointer” refers to a hand-held instrument that is wielded by a user and trackable in the operating room by a tracking system. The “tracked pointer” is configured to aid in the input of data into a computer associated with a computer-assisted surgical system as further described below. In some embodiments, the “tracked pointer” also acts as a “digitizer”, which can measure measuring physical coordinates in three-dimensional space. For example, the “tracked pointer” or “digitizer” may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described for example in U.S. Pat. No. 7,043,961; or an end-effector of a robotic device.

As used herein, the term “digitizing” refers to the collecting, measuring, and/or recording of physical points in space with a digitizer.

As used herein, the term “pre-operative bone data” refers to bone data used to pre-operatively plan a procedure before making modifications to the actual bone. The pre-operative bone data may include one or more of the following. A patients actual exposed bone prior to modification, an image data set of a bone, a virtual generic bone model, a physical bone model, a virtual patient-specific bone model, or a set of data collected directly on a bone intra-operatively commonly used with imageless computer-assist devices.

As used herein, the term “registration” refers to the determination of the POSE and/or coordinate transformation between two or more objects or coordinate systems such as a computer-assist device, a bone, pre-operative bone data, surgical planning data (i.e., an implant model, cut-file, virtual boundaries, virtual planes, cutting parameters associated with or defined relative to the pre-operative bone data), and any external landmarks (e.g., a fiducial marker array) associated with the bone, if such landmarks exist. Methods of registration known in the art are described in U.S. Pat. Nos. 6,033,415, 8,010,177, and 8,287,522.

Also described herein are ‘computer-assisted surgical systems. A computer assisted surgical device refers to any device/system requiring a computer to aid in a surgical procedure. Examples of a computer-assisted surgical device include a tracking system, tracked passive instruments, active or semi-active hand-held surgical devices and systems, autonomous serial-chain manipulator systems, haptic serial chain manipulator systems, parallel robotic systems, or master-slave robotic systems, as described in U.S. Pat. Nos. 5,086,401, 7,206,626, 8,876,830, and 8,961,536, 9,707,043, and PCT. Intl. App. No. US2015/051713.

### Surgical Systems

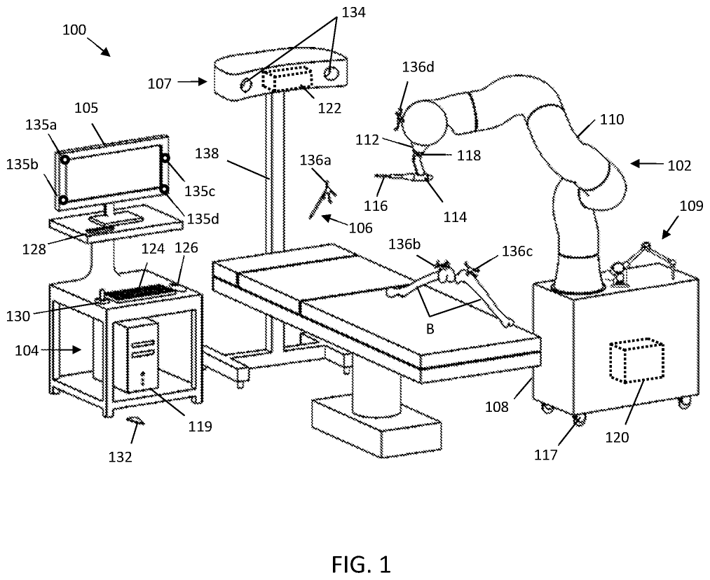

Referring now to the drawings, with reference to FIG. 1, a particular embodiment of a computer-assisted surgical system employing principles of the invention described herein is a robotic surgical system 100. The robotic surgical system 100 generally includes a surgical robot 102, a computing system 104, a tracked display device 105, and a tracked pointer 106 for inputting data into one or more computers of the computing system 104. The surgical system 100 also includes a tracking system 107 including at least one of a mechanical tracking system and/or non-mechanical tracking system (e.g., optical, electromagnetic acoustic). The surgical system 100 may further include a mechanical digitizer 109, which may act as the tracked pointer 106 or be in addition thereto.

The surgical robot 102 may include a movable base 108, a manipulator arm 110 connected to the base 108, an end-effector flange 112 located at a distal end of the manipulator arm 110, and an end-effector assembly 114 for holding and/or operating a tool 116 removably attached to the flange 112 by way of an end-effector mount 118. A force sensor may further be positioned on near the end-effector flange 112 to measure and/or record forces experienced on the tool 116. The base 108 may include an actuation mechanism (e.g., actuator, gears, screws, rails) to adjust the height of the robotic arm 110. The base 108 may further include a set of wheels 117 to maneuver the base 108, which may be fixed into position using a braking mechanism such as a hydraulic brake. The manipulator arm 110 includes various joints and links to manipulate the tool 116 in various degrees of freedom. The joints are illustratively prismatic, revolute, or a combination thereof. The tool 116 may include any surgical tool known in the art including, for example, forceps, endoscope, scissors, clamps, electrocautery, retractor, broach, reamer, rongeur, saw blade, drill bit, or screw. In specific embodiments, the tool 116 is an end-mill adapted to cut bone for orthopedic procedures.

The computing system 104 generally includes a planning computer 119; a device computer 120; a tracking computer 122; and may further include peripheral devices. The planning computer 119, device computer 120, and tracking computer 122, may be separate entities, single units, or combinations thereof depending on the surgical system. The peripheral devices may allow a user to interface with the surgical system components in addition to the user input/feedback accomplished with the tracked display device 105 and tracked pointer 106. The peripheral devices may include: a keyboard 124, mouse 126, pendent 128, joystick 130, foot pedal 132, or the tracked display device 105 in some inventive embodiments has touchscreen capabilities. The tracked display device 105 may include any display known in the art, such as an LED or liquid crystal display (LCD).

The planning computer 119 contains hardware (e.g., processors, controllers, and/or memory), software, data, and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan may include image data, patient data, registration data, implant position data, and/or operational data. The operational data may include: a set of instructions for modifying a volume of tissue that is defined relative to the anatomy, such as a set of cutting parameters (e.g., cut paths, velocities) in a cut-file to autonomously modify the volume of bone; a set of virtual boundaries defined to haptically constrain a tool within the defined boundaries to modify the bone; a set of planes or drill holes to drill pins in the bone; or a graphically navigated set of instructions for modifying the tissue. In particular inventive embodiments, the operational data specifically includes a cut-file for execution by a surgical robot to autonomously modify the volume of bone, which is advantageous from an accuracy and usability perspective. The surgical planning data generated from the planning computer 119 may be transferred to the device computer 120 and/or tracking computer 122 through a wired or wireless connection in the operating room (OR); or transferred via a non-transient data storage medium (e.g., a compact disc (CD), a portable universal serial bus (USB) drive) if the planning computer 119 is located outside the OR. In some embodiments, the surgical plan is transferred via visible light communication as described in U.S. Pat. Pub. No. 2017/0245945 assigned to the assignee of the present application.

The device computer 120 in some inventive embodiments is housed in the moveable base 108 and contains hardware, software, data and utilities that are preferably dedicated to the operation of the surgical robot 102. This may include surgical device control, robotic manipulator control, the processing of kinematic and inverse kinematic data, the execution of registration algorithms, the execution of calibration routines, the execution of operational data (e.g., cut-files), coordinate transformation processing, providing workflow instructions to a user, and utilizing position and orientation (POSE) data from the tracking system 107. In particular embodiments, the device computer 120 is in wired or wireless communication with the tracked display device 105 and the tracking system 107 and may receive input data from the tracking system 107 based on the POSE of the tracked pointer 106 relative to the POSE of the tracked display device 105 as further described below.

The tracking system 107 of the surgical system 100 may be an optical tracking system having two or more optical receivers 134 (e.g., optical cameras) to detect the position of fiducial markers 135 (e.g., retroreflective spheres, active light emitting diodes (LEDs)). The fiducial markers 135 may be uniquely arranged on a rigid body or incorporated directly into a tracked device itself such as the monitor 105. In some embodiments, the fiducial markers 135 are arranged on a rigid body or a device itself, where the collection of markers 135 are collectively referred to as a fiducial marker array 136, such as the array 136a for tracking the digitizer 106. The fiducial markers 135 may by uniquely arranged on the rigid body or tracked device, or have a unique transmitting wavelength/frequency if the markers are active LEDs, to distinguish one tracked device from another. An example of an optical tracking system is described in U.S. Pat. No. 6,061,644. The tracking system 107 may be built into a surgical light, located on a boom, a stand 138, or built into the walls or ceilings of the OR. The tracking system computer 122 may include tracking hardware, software, data and utilities to determine the POSE of objects (e.g., bones B, surgical device 102) in a local or global coordinate frame. The POSE of the objects is collectively referred to herein as POSE data, where this POSE data may be communicated to the device computer 120 through a wired or wireless connection. Alternatively, the device computer 120 may determine the POSE data using the position of the fiducial markers 135 detected from the optical receivers 134 directly.

The POSE data may be determined using the position data detected from the optical receivers 134 and operations/processes such as image processing, image filtering, triangulation algorithms, geometric relationship processing, registration algorithms, calibration algorithms, and coordinate transformation processing. For example, the POSE of the tracked pointer 106 with an attached probe fiducial marker array 136a may be calibrated such that the probe tip is continuously known as described in U.S. Pat. No. 7,043,961. The POSE of the tool tip or tool axis of the tool 116 may be known with respect to a device fiducial marker array 136d using a calibration method as described in U.S. Prov. Pat. App. 62/128,857 (now U.S. Non-prov. patent application Ser. No. 15/548,138) assigned to the assignee of the present application and incorporated by reference herein. It should be appreciated that even though the device fiducial marker 136d is depicted on the manipulator arm 110, it may also be positioned on the base 108 or the end-effector assembly 114. Registration algorithms may be executed to determine the POSE and coordinate transforms between a bone B, pre-operative bone data, a bone fiducial marker array 136b or 136c, and a surgical plan, using the registration methods described above.

The POSE data is used by the computing system 104 during the procedure to update the POSE and/or coordinate transforms of the bone B, the surgical plan, and the surgical robot 102 as the manipulator arm 110 and/or bone B move during the procedure, such that the surgical robot 102 can accurately execute a surgical plan. In another embodiment, the surgical system 100 employs a bone fixation and monitoring system that fixes the bone directly to the surgical robot 102 and monitors bone movement as described in U.S. Pat. No. 5,086,401 without using an optical tracking system wherein the tracked display device 105, and the tracked pointer 106 and/or digitizer 109 are tracked mechanically. The bones may likewise be tracked mechanically in some embodiments.

The POSE data is further used to provide input data to one or more computers associated with the surgical system based on the POSE of the tracked pointer 106 and the tracked display device 105 as further described below.

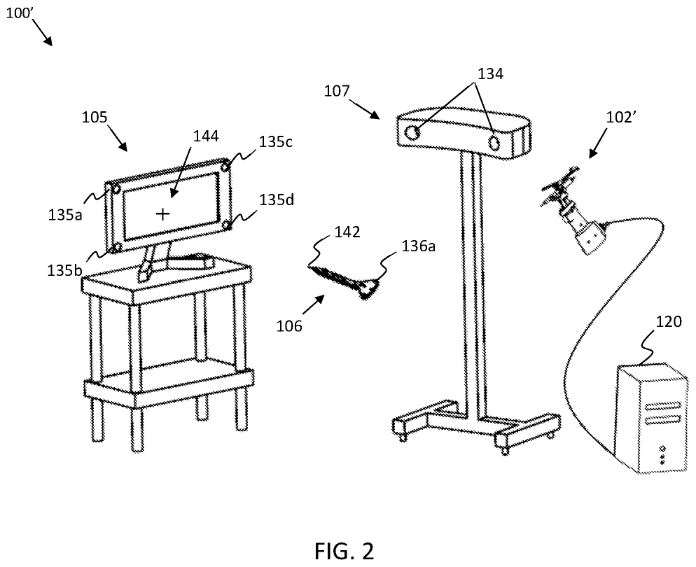

With reference to FIG. 2, a particular embodiment of a computer-assisted surgical system 100 ‘is shown. Here, the surgical system 100’ includes a tracked hand-held surgical device 102′, a device computer 120, tracking system 107, a tracked display device 105, and a tracked pointer 106. The tracked pointer 106 includes a fiducial marker array 136a and a pointer tip 142. The pointer tip 142 designates the pointing direction and may further aid in the digitization of points on one or more objects in the OR. In a particular embodiment, a cursor 144 is displayed on the display device 105 corresponding to the aim of the pointer tip 142 on the display device 105 as determined by the tracking system 107 from the relative POSEs of the pointer 106 and the display device 105. The tracked display device 105 includes a plurality of fiducial markers (135a, 135b, 135c, 135d) directly incorporated into the display device 105 in known positions therewith. The tracked hand-held surgical device 102′ may be any surgical device including, for example, a broach, a reamer, a drill, a scalpel, or a surgical saw. In specific embodiments, the surgical device 102′ is an actuated hand-held surgical device as described in U.S. Pat. Pub. No. 2018/0344409 assigned to the assignee of the present application.

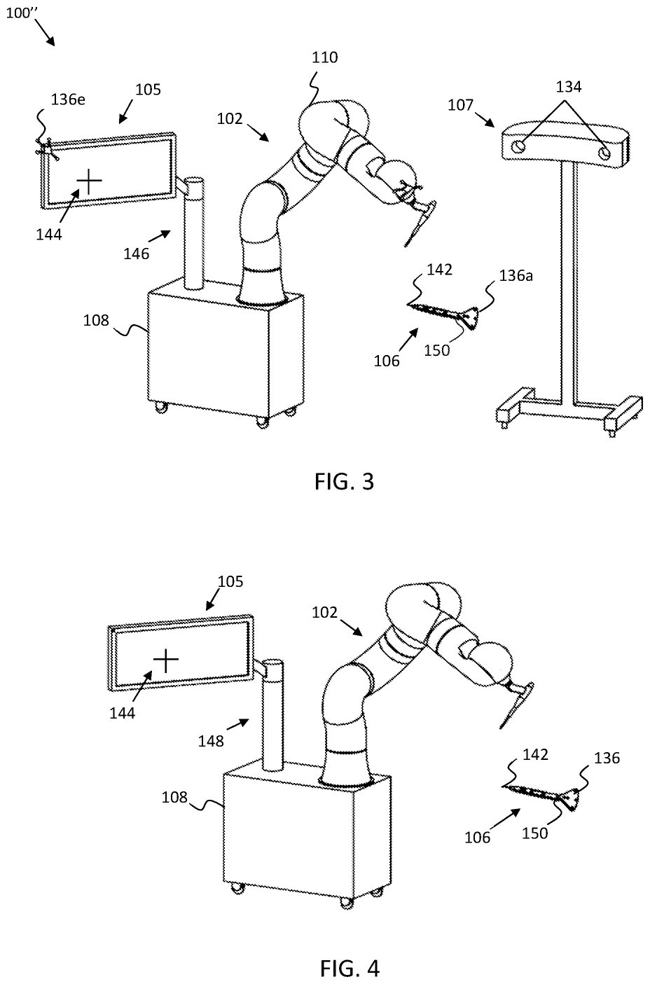

With reference to FIG. 3, a specific embodiment of a computer-assisted surgical system 100″ is shown. Here, the surgical system 100″ includes a surgical robot 102, a tracking system 107, a tracked display device 105, and a tracked pointer 106. The surgical robot 102 is shown having the tracked display device 105 attached to the base 108 of the surgical robot 102. It should be appreciated that the tracked display device 105 may be attached to the robotic arm 110. The display device 105 may be attached to the surgical robot 102 by a first attachment mechanism 146. The first attachment mechanism 146 may be one or more rods. If two or more rods are present, the rods may be attached by joints to permit the user to adjust the position and/or orientation of the display device 105 in the OR. It should further be appreciated that the tracked display device 105 may be attached to the robotic arm 110. In a particular embodiment, the tacked display device 105 is shown having a fiducial marker array 136e attached thereto. This eliminates the need for incorporated fiducials but may require an additional calibration step to accurately track the display device 105. For example, if a fiducial marker array 136e is attached to the display device 105, specific points on the display device 105 may be digitized and matched to corresponding points on a geometric model of the display device 105. Whereas, if fiducial markers (135a, 135b, 135c, 135d) are manufactured directly on the display device 105 in known positions as shown in FIG. 2, the markers are automatically known relative to the geometry of the display device 105.

With reference to FIG. 4, a surgical robot 102 is shown having the tracked display device 105 attached to the base 108 by a mechanical tracking attachment 148. The mechanical tracking attachment 148 may include a plurality of links, joints, and encoders to track the position of the display device 105 if a user adjusts the position and/or orientation of the display device 105 in the OR. Here, if the tracked pointer 106 is also tracked by a mechanical tracking system, there may be no need for a non-mechanical tracking system in the OR, where the bones can be tracked mechanically or rigidly fixed to the robot 102.

In a specific embodiment, the display device 105 is attached to an active attachment mechanism to actively adjust the position and/or orientation of the display device 105 similar to the active trackers as described in U.S. Pat. No. 10,441,366 assigned to the assignee of the present application.

In particular inventive embodiments, the tracked pointer 106 includes one or more selection functions including a button 150, a scroll, or a switch to input data to the computer. The tracked pointer 106 may be connected to one or more computers by a wire connection to communicate the input data from the selection function to the computer. In other embodiments, the tracked pointer 106 is wirelessly connected to one or more computers where the input data is communicated to the computer(s) by way of infrared or visible light as described in U.S. Pat. Pub. No. 2017/0245945 assigned to the assignee of the present application. For example, the pointer 106 may include an active LED for transmitting input data from the selection functions with infrared light to the tracking system 107.

It should be appreciated that the above embodiments, and combinations thereof, permit a user to provide input/feedback to one or more computers of a surgical system in an efficient and effective manner as further described below.

### Data Input/Feedback

During a procedure, the tracked display device 105 may display operational data related to the operation of the surgical device. The data related to the operation of the surgical device may include a set of workflow instructions, prompts, bone models, imaging data, device data, registration instructions, or other procedural data to help a user with the computer-assisted surgical procedure. The user may interact with the operational data on the display device 105 to input data to the computing system using several different methods as described by the following examples. It will be appreciated, that a particular advantage of the systems and methods described herein is the accuracy in interacting with the display device to provide input data. By tracking the POSE of the display device 105, the precise coordinates of each pixel, or neighboring group of pixels, is known to the tracking system 107. As such, the resolution in which the tracked pointer 107 can point to specific areas on the display device 105 is incredibly high, which greatly improves the user's ability to provide input/feedback to the surgical system 100 via the relative POSE of the tracked pointer 106 to the tracked display device 105.

### Example 1

During a surgical procedure, the tracked display device 105 displays a three-dimensional (3-D) model of a bone with a model of an implant thereon as part of a step in the surgical procedure. The display 105 prompts the user to review the POSE of implant model in the bone model to ensure the POSE is as planned. The user, with the tracked pointer 106 in hand, points the pointer tip 142 towards the tracked display device 105. The tracking system 107 detects the pointer tip 142 is pointed towards the display device 105 and activates an input data mode. In the data input mode, the user is capable of interacting with the data on the display device 105 and provide input data to the device computer 120. The user then performs a series of gestures with the tracked pointer 106 to adjust the POSE of the bone model with the implant model therein. For example, the user may perform a swiping gesture with the tracked pointer 106 that the tracking system 107 detects, and in response, the bone model translates in the swiping direction. In another example, the user may gesture a circling motion that the tracking system 107 detects, and in response, the bone model rotates in the circling direction (e.g., clockwise or counterclockwise). One will appreciate the numerous gestures the tracking system 107 may be programmed to detect to manipulate or select data on the display device 105.

After the user has reviewed the POSE of the implant model in the bone model, the user may press a button 150 located on the pointer device 106 to accept or reject the planned POSE of the implant model in the bone model. Alternatively, the user may press the button 150 to activate a cursor mode at which time a cursor 144 is displayed on the display device 105, wherein the position of the cursor 144 accurately matches the aim of the pointer 106 at the display device 105. An accept or reject prompt may be located on the display device 105 where the user can position the cursor on the appropriate response by moving the pointer 106 thereto and selecting the response with a thrust of the pointer 106 towards the display device 105.

Once the user is done interacting with the display device 105, the user points the pointer tip 142 away from the display device 105. In response, the pointer 106 may resume its normal function, if for example the pointer 106 is also a digitizer.

### Example 2

During a surgical procedure, the tracked display device 105 displays a registration routine. To start the registration routine, the display device 105 requests from the user an acknowledgment to begin. The user points the tracked pointer 106 to the display device 105, aims a cursor 144 corresponding to the relative positions therebetween, and presses a button 150 on the tracked pointer 106 to acknowledge the request. Next, the display device 105 displays a plurality of registration points on a bone model for a user to collect on an actual bone. The user then uses the tracked pointer 106 as a digitizer and collects the corresponding points on the bone. Once all of the points have been collected, the user re-points the pointer 106 towards the display device and selects a prompt to signal the completion of point collection. One will appreciate the ease of using the tracked pointer 106 as both a digitizer and input device to quickly interact with the surgical system. Especially since the duration of a surgery is an important factor for any surgical procedure.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A surgical system comprising:
a tracking system:
a surgical device;
a display device; and
a tracked pointer to interact with the display device and input data into a computing system, wherein said tracked pointer inputs data into the computing system based on the relative position and orientation (POSE) of the tracked pointer relative to the display device, wherein said inputted data affects at least one of: displayed data related to a surgical procedure, or the operation of the surgical device itself.

2. The surgical device of claim 1 wherein the displayed data related to the surgical procedure includes at least one of procedural workflow instructions, medical imaging data, three-dimensional anatomical models, or surgical device operating data.

3. The surgical system of claim 1 wherein the tracking system is an optical tracking system with two or more cameras.

4. The surgical system of claim 1 wherein the display device is a tracked display device and the surgical device is a tracked surgical device.

5. The surgical system of claim 18 wherein the second set of fiducial markers are arranged on a rigid body to form a fiducial marker array, wherein the fiducial marker array is attached to the tracked display device.

6. The surgical system of claim 18 wherein the second set of fiducial markers are embedded or integrated with the tracked display device.

7. The surgical system of claim 1 wherein the computing system can only receive input data from the tracked pointer when the tracking system determines the tracked pointer is pointing towards the display device.

8. The system of claim 7 wherein the tracked pointer returns to normal operating conditions when the tracking system determines the tracked pointer is no longer pointed towards the display device.

9. The surgical system of claim 1 wherein the computing system commands the display device to display input indicators that allows the user to input data by pointing to specific areas on the display device.

10. The surgical system of claim 1 wherein the tracked pointer includes one or more selection functions, said one or more selection functions further comprise a button, a scroll, or a switch to select data on the tracked display device.

11. The surgical system of claim 1 wherein the tracking system measures a particular gesture performed by the user with the tracked pointer to generate input data associated with said gesture.

12. The surgical system of claim 11 wherein a particular gesture allows the user to adjust a view of a three-dimensional anatomical model.

13. The surgical system of claim 12 wherein the three-dimensional anatomical model is a bone model.

14. The surgical system of claim 10 wherein the position and orientation of the tracked pointer and the one or more selection functions permit a user to provide input to at least one of: navigate or interact with a surgical workflow, select specific actions related to the surgical workflow, and selecting parameters of the workflow.

15. The surgical system of claim 1 wherein the tracked surgical device is a robotic surgical system having a base and a robotic arm attached thereto, wherein the display device is attached to at least one of the base or the robotic arm.

16. The surgical system of claim 1 wherein the tracked display device is mechanically tracked by one or more links and joints of a mechanical tracking attachment.

17. A method for inputting data into a surgical system comprising:
pointing a tracked pointer towards a display device to activate an input data mode in the computing system;
adjusting at least one of a position or orientation of the tracked pointer to adjust a position of a cursor displayed on the display device; and
selecting one or more selections on the display device by way of: a gesture performed by wielding the tracked pointer, or by activating a selection function on the tracked pointer.

18. The surgical system of claim 4 wherein the tracked pointer includes a first set of fiducial markers and the tracked display device includes a second set of fiducial markers.

19. The method of claim 17 wherein the display device is a tracked display device.
